(19) United States
(12) Patent Application Publication
(10) Pub. No.: US 2004/0258722 A1
(43) Pub. Date: Dec. 23, 2004

(54) Betaine-containing cosmetics

(75) Inventors: Rainer Kropke, Schenefeld (DE); Boris Syskowski, Hamburg (DE); Urte Koop, Hamburg (DE); Andreas Bleckmann, Ahrensburg (DE); Jens Nielsen, Henstedt-Ulzburg (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 10/815,703

(22) Filed: Apr. 2, 2004

Related U.S. Application Data
(63) Continuation of application No. PCT/EP02/10919, filed on Sep. 28, 2002.

(30) Foreign Application Priority Data
Oct. 4, 2001 (DE) 101 48 966.8

(51) Int. Cl. A61K 7/42; A61K 7/00

(52) U.S. Cl. 424/401; 424/59

(57) ABSTRACT

Cosmetic and/or dermatological preparations comprising

a) polyols in a concentration of 5 to 50% by weight,

b) betaines in a concentration of 0.01 to 75% by weight,

in each case based on the total weight of the preparation,

in addition, if appropriate, to other active ingredients, excipients and additives.

### CROSS-REFERENCE TO RELATED APPLICATIONS

The present application is a continuation of International Application No. PCT/EP02/10919, filed Sep. 28, 2002, the entire disclosure whereof is expressly incorporated by reference herein, which claims priority under 35 U.S.C. § 119 of German Patent Application No. 101 48 966.8, filed Oct. 4, 2001.

The present invention relates to cosmetic and/or dermatological preparations which comprise polyols and betaines, and to their use.

The skin is the largest human organ. Among its many functions (for example for heat regulation and as a sense organ), the barrier function, which prevents the drying out of the skin (and thus in the final analysis of the entire body), is probably the most important. At the same time, the skin acts as a protective device against the penetration and the absorption of substances originating from outside. This barrier function is brought about by the epidermis, which as the outermost layer forms the actual protective coat against the environment. At approximately one-tenth of the total thickness, it is, at the same time, the thinnest layer of the skin.

It is the object of cosmetic skincare to strengthen or restore the natural function of the skin as a barrier against environmental influences (e.g. dirt, chemicals, microorganisms) and against the loss of endogenous substances (e.g. water, natural fats, electrolytes).

The aim of skincare is furthermore to compensate the fat loss of the skin caused by daily washing. This is particularly important if the natural capacity for regeneration is not adequate.

One of the most important objects of skincare, however, consists in moisturizing the skin. The classic skin moisturizing agents include, inter alia, polyols such as glycerol and sorbitol. In addition, however, other compounds are also employed, such as ethoxylated polyols and hydrolyzed proteins. Moreover, components of the natural moisturizing factor of the skin (NMF), e.g. urea and certain amino acids, are used. Glycerol, however, is most widespread. A great disadvantage in the prior art consists in the negative sensory properties of polyol-containing cosmetic and/or dermatological preparations. As a rule, these feel sticky and greasy on the skin and make the corresponding products unattractive to the consumer.

It was therefore the object of the present invention to eliminate or to decrease the disadvantages of the prior art and to develop polyol-containing cosmetic and/or dermatological preparations whose sticky and greasy skin sensation is reduced.

Surprisingly, the object is achieved by cosmetic and/or dermatological preparations comprising

a) polyols in a concentration of 5 to 50% by weight,

b) betaines in a concentration of 0.01 to 75% by weight,

in each case based on the total weight of the preparation,

in addition, if appropriate, to other active ingredients, excipients and additives.

A concentration of polyols of 5 to 20% by weight and in particular of 5 to 15% by weight is particularly advantageous, in each case based on the total weight of the formulation.

A concentration of betaines of 0.5 to 50% by weight and in particular of 3 to 15% by weight is furthermore particularly advantageous, in each case based on the total weight of the formulation.

These preparations are distinguished by a pleasant less sticky less greasy skin sensation without the good skin-moisturizing properties being lost.

The betaine particularly preferred according to the invention is in this case trimethyl-glycine; as preferred polyol according to the invention glycerol is employed.

Part of the invention is the use of betaines for reducing the stickiness of polyol-containing cosmetic and/or dermatological preparations.

Part of the invention is furthermore the use of betaines for reducing the greasy skin sensation of polyol-containing cosmetic and/or dermatological preparations.

Generally, the use of betaines for improving the sensory properties of cosmetic and/or dermatological preparations according to the invention is novel.

Furthermore, the use of betaines in cosmetic and/or dermatological preparations after for reducing the adherence of foreign particles, in particular sand, to skin treated with cosmetic and/or dermatological preparations is novel.

The novel cosmetic and/or dermatological preparations can be employed according to the invention as a skin cream, face cream and/or body lotion, and as a sunscreen.

It is, of course, known to the person skilled in the art that demanding cosmetic compositions are usually not conceivable without the customary excipients and additives. The novel cosmetic preparations can therefore contain cosmetic excipients, such as are customarily used in such preparations, e.g. preservatives, bactericides, perfumes, vitamins, colorants, agents for preventing foaming, thickeners, plasticizing substances, fats, oils, waxes or other customary constituents of a cosmetic formulation such as alcohols, polymers, foam stabilizers, electrolytes or organic solvents.

Thus it is advantageous according to the invention to add UV filters to the novel preparations. Here, oil-soluble UVA filters and/or UVB filters can be employed in the lipid phase and/or water-soluble UVA filters and/or UVB filters can be employed in the aqueous phase.

Advantageous oil-soluble UVB filter substances are, for example:

4-aminobenzoic acid derivatives, preferably 2-ethylhexyl 4-(dimethylamino)-benzoate, amyl 4-(dimethylamino)benzoate;

esters of benzalmalonic acid, preferably 2-ethylhexyl 4-methoxybenzal-malonate;

esters of cinnamic acid, preferably 2-ethylhexyl methoxycinnamate, isopentyl 4-methoxycinnamate;

derivatives of benzophenone, preferably 2-hydroxy-4-methoxybenzophenone, 2-hydroxy-4-methoxy-4′-methylbenzophenone, 2,2′-dihydroxy-4-methoxy-benzophenone;

methylidenecamphor derivatives, preferably 4-methylbenzylidenecamphor, benzylidenecamphor,

symmetrically substituted triazines, preferably tris(2-ethylhexyl) 4,4′,4″-(1,3,5-triazine-2,4,6-triyltriimino)trisbenzoate, synonym: 2,4,6-tris[anilino(p-carbo-2′-ethyl-1′-hexyloxy)]-1,3,5-triazine [UVINUL T 150 (BASF)],

unsymmetrically substituted triazines, preferably 2,4-bis{[4-(2-ethylhexyloxy)-2-hydroxy]phenyl}-6-(4-methoxyphenyl)-1,3,5-triazine (INCI: Aniso triazine) and 2,4-bis(2-ethylhexyl p-aminobenzoate)-6-(p-aminobenzoic acid N-1,1-di-methylethylamide)-1,3,5-triazine dioctylbutylamidotriazone (INCI: dioctylbutylamidotriazone).

Advantageous water-soluble UVB filter substances are, for example:

sulfonic acid derivatives of 3-benzylidenecamphor, such as, for example, 4-(2-oxo-3-bornylidenemethyl)benzenesulfonic acid, 2-methyl-5-(2-oxo-3-bornylidene)sulfonic acid and their salts,

2-phenylbenzimidazole-5-sulfonic acid and its salts [Eusolex 232 (Merck), Neo Heliopan Hydro (H & R), Parsol HS (Givaudan)].

The list of UVB filters mentioned, which can additionally be employed within the meaning of the present invention, should, of course, be non-limiting.

Advantageous UVA filters are, for example:

1,4-phenylenedimethinecamphorsulfonic acid derivatives such as, for example, 3,3′-(1,4-phenylenedimethine)bis(7,7-dimethyl-2-oxobicyclo[2.2.1]-heptane-1-methanesulfonic acid and its salts

dibenzoylmethane derivatives, preferably 4-isopropyldibenzoylmethane, 4-(tert-butyl)-4′-methoxydibenzoylmethane

phenylene-1,4-bis(2-benzimidazyl)-3,3′,5,5′-tetrasulfonic acid and its sodium, potassium and triethanolammonium salts, in particular the disodium salt [Neo Heliopan AP (Haarmann & Reimer)].

Furthermore, it can optionally be advantageous according to the invention to provide the preparations with further UVA and/or UVB filters, for example certain salicylic acid derivatives such as 4-isopropylbenzyl salicylate, 2-ethylhexyl salicylate, octyl salicylate and homomenthyl salicylate.

Further light screen filter substances to be advantageously used according to the invention are ethylhexyl 2-cyano-3,3-diphenyl acrylate (octocrylene), which is obtainable from BASF under the name UVINUL® N 539, and 2,2′-methylene-bis(6-(2H-benzotriazol-2-yl)-4-(1,1,3,3-tetramethylbutyl)phenol).

These further UV light screen filters which are advantageous according to the invention are preferably employed in a concentration of from 0.1 to 20% by weight, in particular in a concentration of from 0.5 to 10% by weight, based on the total weight of the formulation.

According to the invention, the cosmetic and/or dermatological light screen formulations can have the customary composition and can be used for cosmetic and/or dermatological light protection, further for the treatment, the care and the cleansing of the skin and/or the hair and as a makeup in decorative cosmetics.

For use, the cosmetic and/or dermatological preparations are applied according to the invention to the skin and/or the hair in sufficient amount in the manner customary for cosmetics.

The cosmetic and/or dermatological preparations can, according to the invention, contain pigments (e.g. TiO₂, ZnO, BaSO₄) which have a coloring action and/or can be employed advantageously according to the invention as inorganic light screen filters. Further excipients according to the invention are fats, oils, waxes or other customary constituents of a cosmetic or dermatological formulation such as alcohols, polymers, foam stabilizers, electrolytes, organic solvents or silicone derivatives.

An additional content of antioxidants is in general preferred. According to the invention, favorable antioxidants which can be used are all antioxidants suitable or customary for cosmetic and/or dermatological applications.

Advantageously, the antioxidants are chosen from the group consisting of amino acids (e.g. glycine, histidine, tyrosine, tryptophan) and their derivatives, imidazoles (e.g. urocaninic acid) and their derivatives, peptides such as D,L-carnosine, D-carnosine, L-carnosine and their derivatives (e.g. anserine), carotenoids, carotenes (e.g. α-carotene, β-carotene, lycopene) and their derivatives, chlorogenic acid and its derivatives, lipoic acid and its derivatives (e.g. dihydrolipoic acid), aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters), and their salts, dilauryl thiodipropionate, distearyl thiodipropionate, thiodipropionic acid and its derivatives (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerable doses (e.g. pmol to μmol/kg), furthermore (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. γ-linolenic acid, linoleic acid, oleic acid), folic acid and its derivatives, ubiquinone and ubiquinol and their derivatives, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutic acid and its derivatives, α-glycosylrutin, ferulic acid, furfurylideneglucitol, carnosine, butylhydroxytoluene, butylhydroxyanisole, nordihydroguaiaretic acid, nordihydroguaiaretic acid, trihydroxybutyrophenone, uric acid and its derivatives, mannose and its derivatives, zinc and its derivatives (e.g. ZnO, ZnSO₄) selenium and its derivatives (e.g. selenomethionine), stilbenes and their derivatives (e.g. stilbene oxide, trans-stilbene oxide) and the derivatives suitable according to the invention (salts, esters, ethers, sugars, nucleotides, nucleosides, peptides and lipids) of these said active compounds.

The amount of the abovementioned antioxidants (one or more compounds) in the preparations is preferably 0.001 to 10% by weight, particularly preferably 0.05-7% by weight, in particular 0.5-5% by weight, based on the total weight of the preparation.

If vitamin E and/or its derivatives is/are the antioxidant(s), their respective concentrations are advantageously to be chosen from the range from 0.001-5% by weight, based on the total weight of the formulation.

If vitamin A or vitamin A derivatives, or carotenes or their derivatives is/are the antioxidant(s), it is advantageous to choose their respective concentrations from the range of 0.001-5% by weight, based on the total weight of the formulation.

Moreover, selected recipes according to the invention, which, for example, contain known antiwrinkle active ingredients such as flavone glycosides (in particular α-glycosylrutin), coenzyme Q10, vitamin E and/or derivatives and the like, are suitable, in particular advantageous, for the prophylaxis and treatment of cosmetic or dermatological skin changes, such as occur, for example, during skin ageing (e.g. wrinkles and folds). They are furthermore advantageously suitable against the syndrome of dry or rough skin.

An optionally present lipid phase can advantageously be chosen from the following substance group:

mineral oils, mineral waxes

oils, such as triglycerides of capric or caprylic acid, but preferably castor oil;

fats, waxes and other natural and synthetic fatty substances, preferably esters of fatty acids with alcohols of low C number, e.g. with isopropanol, propylene glycol or glycerol, or esters of fatty alcohols with alkanoic acids of low C number or with fatty acids;

alkyl benzoates;

silicone oils such as dimethylpolysiloxanes, diethylpolysiloxanes, diphenylpolysiloxanes and mixed forms thereof.

An oil phase of the emulsions, oleogels or hydrodispersions or lipodispersions within the meaning of the present invention is advantageously chosen from the group consisting of the esters of saturated and/or unsaturated, branched and/or unbranched alkanecarboxylic acids of a chain length of 3 to 30 C atoms and saturated and/or unsaturated, branched and/or unbranched alcohols of a chain length of 3 to 30 C atoms, from the group consisting of the esters of aromatic carboxylic acids and saturated and/or unsaturated, branched and/or unbranched alcohols of a chain length of 3 to 30 C atoms. Such ester oils can then be advantageously chosen from the group consisting of isopropyl myristate, isopropyl palmitate, isopropyl stearate, isopropyl oleate, n-butyl stearate, n-hexyl laurate, n-decyl oleate,

isooctyl stearate, isononyl stearate, isononyl isononanoate, 2-ethyl-hexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

Furthermore, the oil phase can advantageously be chosen from the group consisting of the branched and unbranched hydrocarbons and hydrocarbon waxes, of the silicone oils, of the dialkyl ethers, of the group consisting of the saturated or unsaturated, branched or unbranched alcohols, and of the fatty acid triglycerides, especially the triglycerol esters of saturated and/or unsaturated, branched and/or unbranched alkanecarboxylic acids of a chain length of 8 to 24, in particular 12-18, C atoms. The fatty acid triglycerides can, for example, advantageously be chosen from the group consisting of the synthetic, semisynthetic and natural oils, e.g. olive oil, sunflower oil, soybean oil, groundnut oil, rapeseed oil, almond oil, palm oil, coconut oil, palm kernel oil and suchlike.

Any desired mixtures of such oil and wax components are also advantageously to be employed within the meaning of the present invention. It can also optionally be advantageous to employ waxes, for example cetyl palmitate, as the sole lipid component of the oil phase.

Advantageously, the oil phase is chosen from the group consisting of 2-ethylhexyl isostearate, octyidodecanol, isotridecyl isononanoate, isoeicosane, 2-ethylhexyl cocoate, C₁₂₋₁₅-alkyl benzoate, caprylic/capric acid triglyceride, dicaprylyl ether, di-caprylyl carbonate, butylene glycol dicaprylate/dicaprate.

Mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate are particularly advantageous.

Of the hydrocarbons, paraffin oil, squalane and isoparaffin are to be used advantageously within the meaning of the present invention.

Advantageously, the oil phase can further contain cyclic or linear silicone oils or consist completely of such oils, it being preferred, however, to use an additional content of other oil phase components apart from the silicone oil or the silicone oils.

Advantageously, cyclomethicone (octamethylcyclotetrasiloxane) or dimethicone is employed as the silicone oil to be used according to the invention. However, other silicone oils are also to be used advantageously within the meaning of the present invention, for example hexamethylcyclotrisiloxane, polydimethylsiloxane, poly-(methylphenylsiloxane).

Mixtures of cyclomethicone and isotridecyl isononanoate, of cyclomethicone and 2-ethylhexyl isostearate are furthermore particularly advantageous.

The aqueous phase of the novel preparations optionally advantageously contains

alcohols, diols or polyols of low C number, and their ethers, preferably ethanol, isopropanol, propylene glycol, glycerol, ethylene glycol, ethylene glycol monoethyl or monobutyl ether, propylene glycol monomethyl, mono-ethyl or monobutyl ether, diethylene glycol monomethyl or monoethyl ether and analogous products, furthermore alcohols of low C number, e.g. ethanol, isopropanol, 1,2-propanediol, glycerol and in particular one or more thickening agents, which can advantageously be chosen from the group consisting of silica, aluminum silicates, polysaccharides and their derivatives, e.g. hyaluronic acid, xanthan gum, hydroxypropylmethylcellulose, particularly advantageously from the group consisting of the polyacrylates, preferably a polyacrylate from the group consisting of the “carbopols”, for example carbopols of the types 980, 981, 1382, 2984, 5984, in each case individually or in combination.

The cosmetic and/or dermatological preparations according to the invention can be employed advantageously according to the invention in O/W emulsions in and in emulsifier-free O/W emulsions (hydrodispersions).

The following examples are intended to illustrate the present invention without restricting it. All quantitative data, proportions and percentages are, if not stated otherwise, based on the weight and the total amount or on the total weight of the emulsion.

W/O emulsions

| | 1 | 2 | 3 | 4 | 5 |
|---|---|---|---|---|---|
| Triglycerol diisostearate | 1.0 | 0.5 | 0.25 | 2.0 | 3.0 |
| Diglycerol dipolyhydroxystearate | 1.0 | 1.5 | 1.75 | 3.0 | 2.0 |
| Paraffin oil | 12.5 | 10.0 | 8.0 | 5.0 | 7.5 |
| Petroleum jelly | 8.0 | 6.0 | 5.0 | 12.0 | 2.5 |
| Hydrogenated coconut glycerides | 2.0 | 1.0 | 2.5 | 5.0 | 0.25 |
| Decyl oleate | 0.5 | 0.75 | 1.0 | 2.0 | 0.25 |
| Octyldodecanol | 0.5 | 1.0 | 0.75 | 3.0 | 0.25 |
| Aluminum stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Betaine | 5 | 2.5 | 12.5 | 7.5 | 22 |
| Magnesium sulfate | 0.5 | 0.6 | 0.5 | 0.7 | 1.0 |
| Glycerol | — | 5.0 | — | 15.0 | 5 |
| Sorbitol | 15 | — | 5 | — | — |
| Citric acid | 0.2 | 0.1 | 0.2 | 0.3 | 1.0 |
| Perfume | q.s. | q.s. | q.s. | q.s. | q.s. |
| Ethanol | 2.0 | — | 5.0 | — | — |
| Caprylic/capric acid triglyceride | 2.0 | 2.5 | 3.0 | 5.0 | 0.5 |
| Methylparaben | 0.4 | 0.15 | 0.05 | 0.3 | 0.4 |
| Propylparaben | 0.3 | 0.4 | 0.25 | 0.15 | — |
| Iodopropynylbutyl carbamate | — | — | 0.05 | — | 0.1 |
| Water | to 100 | to 100 | to 100 | to 100 | to 100 |
| | 6 | 7 | 8 | 9 | 10 |
| PEG-30 dipolyhydroxystearate | — | 0.5 | 0.25 | — | 3.0 |
| Lanolin alcohol | 1.0 | 1.5 | 1.75 | 3.0 | — |
| Paraffin oil | 12.5 | 10.0 | 8.0 | 5.0 | 17.5 |
| Petroleum jelly | 8.0 | 6.0 | 5.0 | 2.0 | 2.5 |
| Hydrogenated coconut glycerides | 2.0 | 1.0 | 2.5 | 5.0 | 0.25 |
| Hydrogenated polyisobutene | 0.5 | 0.75 | 1.0 | 2.0 | 0.25 |
| Octyldodecanol | 0.5 | 1.0 | 0.75 | 3.0 | 0.25 |
| Aluminum stearate | 0.4 | 0.3 | 0.6 | 1.0 | 0.05 |
| Dicaprylyl carbonate | 0.1 | 0.05 | 0.15 | 0.5 | 1.0 |
| Hydrogenated castor oil | 0.5 | 0.75 | 1.0 | 2.5 | 5.0 |
| Betaine | 2.5 | 12.5 | 25 | 35 | 7.5 |
| Magnesium sulfate | 0.5 | 0.6 | 0.5 | 0.7 | 1.0 |
| Glycerol | 3.0 | 5.0 | — | — | — |
| Sorbitol | — | — | 5 | 12 | 25 |
| Citric acid | 0.2 | 0.1 | 0.2 | 0.3 | 1.0 |
| Perfume | q.s. | q.s. | q.s. | q.s. | q.s. |
| 1,3-butylene glycol | 2.0 | — | 5.0 | — | — |
| Caprylic/capric acid triglyceride | 2.0 | 2.5 | 3.0 | 5.0 | 0.5 |
| Methylparaben | 0.4 | 0.15 | 0.05 | 0.3 | 0.4 |
| Propylparaben | 0.3 | 0.4 | 0.25 | 0.15 | — |
| Water | to 100 | to 100 | to 100 | to 100 | to 100 |

W/S emulsion

| | 11 | 12 | 13 | 14 | 15 |
|---|---|---|---|---|---|
| Cetyldimethicone copolyol | 1.0 | — | — | 3.0 | 5.0 |
| Cyclomethicone + PEG/PPG-18/18 | 10.0 | 12.5 | 25 | — | — |
| dimethicone | | | | | |
| Cyclomethicone | 12.5 | 15 | 8 | 25.0 | 17.5 |
| Dimethicone | 5.0 | 13.0 | 5.0 | 12.0 | 15.0 |
| Hydrogenated polyisobutyene | 0.5 | 0.75 | 1.0 | 2.0 | 0.25 |
| Octyldodecanol | 0.5 | 1.0 | 0.75 | 3.0 | 0.25 |
| Panthenol | 0.5 | 1.0 | 0.75 | 0.25 | 0.1 |
| Sodium chloride | 2.0 | 0.6 | 2.5 | 0.7 | 1.0 |
| Glycerol | 3.0 | — | — | — | 15 |
| Sorbitol | — | 5 | 15 | 7.5 | — |
| Citric acid | 0.2 | 0.1 | 0.2 | 0.3 | 1.0 |
| Perfume | q.s. | q.s. | q.s. | q.s. | q.s. |
| Methylparaben | 0.4 | 0.1 | 0.05 | 0.3 | 0.4 |
| Betaine | 12.5 | 5 | 45 | 25 | 3 |
| Propylparaben | 0.3 | 0.4 | 0.25 | 0.15 | — |
| Cetyldimethicone | 0.5 | — | 0.7 | — | — |
| Iodopropynylbutyl carbamate | — | — | 0.05 | — | 0.1 |
| Modified starch | — | 2.5 | — | 0.15 | — |
| Water | to 100 | to 100 | to 100 | to 100 | to 100 |

W/S emulsions

| | 16 | 17 | 18 | 19 | 20 |
|---|---|---|---|---|---|
| Cetyldimethicone copolyol | 1.0 | — | — | 3.0 | 5.0 |
| Cyclomethicone + PEG/PPG-18/18 | 10.0 | 12.5 | 25 | — | — |
| dimethicone | | | | | |
| Cyclomethicone | 12.5 | 15 | 28.0 | 25.0 | 17.5 |
| Dimethicone | 5.0 | 13.0 | 5.0 | 12.0 | 15.0 |
| Hydrogenated polyisobutyene | 0.5 | 0.75 | 1.0 | 2.0 | 0.25 |
| Octyldodecanol | 0.5 | 1.0 | 0.75 | 3.0 | 0.25 |
| Panthenol | 0.5 | 1.0 | 0.75 | 0.25 | 0.1 |
| Sodium chloride | 2.0 | 0.6 | 2.5 | 0.7 | 1.0 |
| Sorbitol | 3.0 | 5.0 | 10.0 | 15.0 | 1.5 |
| Citric acid | 0.2 | 0.1 | 0.2 | 0.3 | 1.0 |
| Perfume | q.s. | q.s. | q.s. | q.s. | q.s. |
| Methylparaben | 0.4 | 0.1 | 0.05 | 0.3 | 0.4 |
| Betaine | 10 | 20 | 25 | 7.45 | 3.25 |
| Propylparaben | 0.3 | 0.4 | 0.25 | 0.15 | — |
| Stearyldimethicone | 0.5 | — | 0.7 | — | — |
| Iodopropynylbutyl carbamate | — | — | 0.05 | — | 0.1 |
| Modified starch | — | 2.5 | — | 0.15 | — |
| Water | to 100 | to 100 | to 100 | to 100 | to 100 |

Oil-in-water emulsion

| | 21 | 22 | 23 | 24 | 25 |
|---|---|---|---|---|---|
| Glyceryl stearate | 1.0 | — | — | 3.0 | 5.0 |
| PEG-40 stearate | 10.0 | — | 5 | — | — |
| Triglycerol methylglucose distearate | — | 5.5 | — | — | 2.5 |
| Sorbitan stearate | — | 1.5 | 3 | — | — |
| Cyclomethicone | 1 | 2.5 | 5 | 7.5 | 3 |
| Dimethicone | 5.0 | 13.0 | 5.0 | 12.0 | 15.0 |
| Behenyl alcohol | 1 | | 2 | 1 | — |
| Stearyl alcohol | | 1 | | 1 | — |
| Cetylstearyl alcohol | | | 1 | 1 | — |
| Hydrogenated polyisobutene | 0.5 | 0.75 | 1.0 | 2.0 | 0.25 |
| Octyldodecanol | 0.5 | 1.0 | 0.75 | 3.0 | 0.25 |
| Betaine | 5 | 7.5 | 10 | 15 | 25 |
| Panthenol | 0.5 | 1.0 | 0.75 | 0.25 | 0.1 |
| Sorbitol | — | — | 3.0 | 5.0 | 12.5 |
| Glycerol | 3.0 | 5.0 | — | — | — |
| Perfume | q.s. | q.s. | q.s. | q.s. | q.s. |
| Methylparaben | 0.4 | 0.1 | 0.05 | 0.3 | 0.4 |
| Propylparaben | 0.3 | 0.4 | 0.25 | 0.15 | — |
| Iodopropynylbutyl carbamate | — | — | 0.05 | — | 0.1 |
| Modified starch | — | 2.5 | — | 0.15 | — |
| Water | to 100 | to 100 | to 100 | to 100 | to 100 |
| | 26 | 27 | 28 | 29 | 30 |
| Polyethylene glycol (21) stearyl ether | 1 | — | 2.5 | 2 | 1.5 |
| Polyethylene glycol (2) stearyl ether | 1 | — | 5.5 | 3 | 7.5 |
| Cetearyl glucoside | — | 8 | — | — | — |
| Cyclomethicone | 2.5 | 3 | 12.5 | 2 | — |
| Dimethicone | 5.0 | 13.0 | 5.0 | 12.0 | 15.0 |
| Behenyl alcohol | 3 | 2 | — | 1 | — |
| Stearyl alcohol | 3 | 2 | | 2 | — |
| Cetylstearyl alcohol | 3 | 4 | — | — | 2 |
| Hydrogenated polyisobutene | 0.5 | 0.75 | 1.0 | 2.0 | 0.25 |
| Octyldodecanol | 0.5 | 1.0 | 0.75 | 3.0 | 0.25 |
| Betaine | 5 | 7.5 | 10 | 15 | 25 |
| Sorbitol | — | — | 7.5 | 12.5 | 10 |
| Panthenol | 0.5 | 1.0 | 0.75 | 0.25 | 0.1 |
| Glycerol | 3.0 | 5.0 | — | — | — |
| Perfume | q.s. | q.s. | q.s. | q.s. | q.s. |
| Methylparaben | 0.4 | 0.1 | 0.05 | 0.3 | 0.4 |
| Propylparaben | 0.3 | 0.4 | 0.25 | 0.15 | — |
| Iodopropynylbutyl carbamate | — | — | 0.05 | — | 0.1 |
| Modified starch | — | 2.5 | — | 0.15 | — |
| Water | to 100 | to 100 | to 100 | to 100 | to 100 |

1.-9. (canceled).

10. A cosmetic or dermatological composition comprising:
(a) from 5% to 50% by weight of one or more polyols;
(b) from 0.01% to 75% by weight of one or more betaines.

11. The composition of claim 10, wherein the one or more polyols comprise at least one of glycerol and sorbitol.

12. The composition of claim 11, wherein the composition comprises from 5% to 20% by weight of the one or more polyols.

13. The composition of claim 10, wherein the composition comprises from 5% to 15% by weight of the one or more polyols.

14. The composition of claim 10, wherein the one or more polyols comprise glycerol.

15. The composition of claim 10, wherein the one or more betaines comprise trimethylglycine.

16. The composition of claim 14, wherein the one or more betaines comprise trimethylglycine.

17. The composition of claim 10, wherein the composition comprises from 0.5% to 50% by weight of the one or more betaines.

18. The composition of claim 17, wherein the composition comprises up to 15% by weight of the one or more betaines.

19. The composition of claim 17, wherein the composition comprises at least 3% by weight of the one or more betaines.

20. The composition of claim 13, wherein the composition comprises from 3% to 15% by weight of the one or more betaines.

21. The composition of claim 10, wherein the composition further comprises a UV filter substance.

22. The composition of claim 21, wherein the UV filter substance comprises at least one of a UVA and a UVB filter substance.

23. The composition of claim 10, wherein the composition further comprises a pigment.

24. The composition of claim 23, wherein the pigment comprises at least one of TiO₂, ZnO and BaSO₄.

25. The composition of claim 10, wherein the composition further comprises one or more antioxidants.

26. The composition of claim 25, wherein the one or more antioxidants are present in a concentration of from 0.05% to 7% by weight.

27. The composition of claim 10, wherein the composition comprises:
(a) from 5% to 20% by weight of one or more polyols which comprise at least one of sorbitol and glycerol;
(b) from 0.5 to 50% by weight of one or more betaines which comprise trimethylglycine.

28. The composition of claim 27, wherein the composition comprises up to 15% by weight of the one or more polyols.

29. The composition of claim 28, wherein the composition comprises from 3% to 15% by weight of the one or more betaines.

30. The composition of claim 29, wherein the one or more polyols comprise glycerol.

31. An emulsion which comprises the composition of claim 10.

32. The emulsion of claim 31, wherein the emulsion comprises an O/W emulsion.

33. The emulsion of claim 31, wherein the emulsion comprises a W/O emulsion.

34. A body lotion which comprises the composition of claim 10.

35. A face cream which comprises the composition of claim 10.

36. A skin cream which comprises the composition of claim 10.

37. A sunscreen which comprises the composition of claim 10.

38. A method of reducing a stickiness of a polyol-containing cosmetic or dermatological composition, wherein the method comprises incorporating into the composition one or more betaines.

39. A method of reducing a greasy skin sensation of a polyol-containing cosmetic or dermatological composition, wherein the method comprises incorporating into the composition one or more betaines.

40. A method of improving sensory properties of a cosmetic or dermatological composition, wherein the method comprises incorporating into the composition one or more betaines.

41. A method of reducing adherence of foreign particles to skin treated with a cosmetic or dermatological composition, wherein the method comprises incorporating into the composition one or more betaines.

42. The method of claim 41, wherein the foreign particles comprise sand particles.
